(19) United States
(12) Patent Application Publication
(10) Pub. No.: US 2003/0180264 A1
(43) Pub. Date: Sep. 25, 2003

(54) Devices and methods for delivery of a bioactive compound to an organism

(75) Inventors: Herman H. Vandenburgh, Providence, RI (US); Robert F. Valentini, Cranston, RI (US)

Correspondence Address: PALMER & DODGE, LLP, KATHLEEN M. WILLIAMS, 111 HUNTINGTON AVENUE, BOSTON, MA, 02199, US

(73) Assignee: Cell Based Delivery Inc.

(21) Appl. No.: 10/376,539

(22) Filed: Feb. 28, 2003

Related U.S. Application Data
(63) Continuation of application No. 09/343,990, filed on Jun. 29, 1999, now abandoned.

(51) Int. Cl. A61K 35/12; C12N 5/06

(52) U.S. Cl. 424/93.7; 604/522; 435/1.1

(57) ABSTRACT

A method and device for delivery of a tissue to an organism uses a catheter or stent to deliver organized tissue to an anatomic site. Organized tissue may, in certain preferred embodiments, be grown in vitro in a catheter or stent, or in vitro independent of the catheter or stent, and then transferred to the catheter or stent. The organized tissue is transported to a desired anatomic site in vivo via catheter or stent. The organized tissue may be retrieved from the anatomic site to terminate treatment.

### FIELD OF THE INVENTION

The present invention is directed to a device and method for delivery of a tissue to an anatomic site in an organism.

### BACKGROUND

It is known that diseases can be treated via production of therapeutic proteins via genetic engineering of cells and tissues. For example, myoblast stem cells genetically engineered to secrete erythropoietin can be used to treat anemia when injected intramuscularly as described in Persistent Erythropoiesis By Myoblast Transfer of Erythropoietin cDNA, Fang, J. et al., Proc. Natl. Acad. Sci. USA 93: pages 5753-5758. However, millions of proliferating and migratory myoblasts must be injected at muscle bed sites with poor prediction of cell survival and protein delivery dose. In another example, in vivo cells may be used for cell therapy by injecting plasmid DNA into bone tissue, but dose and localization of the recombinant protein secreting cells is difficult as described in Stimulation of New Bone Formation By Direct Transfer of Osteogenic Plasmid Genes, Hamamori et al., Human Gene Therapy 5: pages 1349-1356. Both procedures are also irreversible. Tissue engineered products such as skin can also be genetically engineered to secrete therapeutic proteins and transplanted to host skin as described in Expression of an Exogenous Growth Hormone Gene By Transplantable Human Epidermal Cells, Morgan et al., Science 237: pages 1476-1479. But these procedures involve elaborate surgical procedures and contain proliferating cells which die within a relatively short time frame, i.e., 4 to 6 weeks.

It is an object of the present invention to deliver organized tissue to an anatomic site and reduce or wholly overcome some or all of the aforesaid difficulties inherent in prior known devices. Particular objects and advantages of the invention will be apparent to those skilled in the art, that is, those who are knowledgeable or experienced in this field of technology, in view of the following disclosure of the invention and detailed description of certain preferred embodiments.

### SUMMARY OF THE INVENTION

The principles of the invention may be used to advantage to provide organized tissue at an anatomic site in an organism using minimally invasive techniques.

In accordance with a first aspect, tissue may be delivered to an organism via a catheter containing organized tissue. In all cases, the catheter can contain an organized tissue alone or in a sleeve or stent.

In accordance with a second aspect, an organized tissue that produces a bioactive compound may be introduced to an anatomic site via a catheter.

In accordance with another aspect, a method of delivering tissue to an organism is performed by providing a catheter containing organized tissue and introducing the organized tissue to an anatomic site in the organism via a catheter.

In accordance with another aspect, a method of delivering a bioactive compound to an organism is performed by providing a catheter, placing organized tissue at a distal end of the catheter and transporting the organized tissue to an anatomic site via the catheter.

In accordance with yet another aspect, a method of delivering a bioactive compound to an organism is performed by providing a catheter, growing organized tissue that produces a bioactive compound within the catheter, and transporting the organized tissue to an anatomic site via the catheter.

In accordance with a further aspect, a kit for delivery of tissue to an organism has a catheter, organized tissue, a culture media and buffer for maintaining the organized tissue, and packaging materials.

As used herein, “organism” refers to a non-mammal or a mammal, including a human.

As used herein, “catheter” refers to a tubular device which allows the passage of fluid and or other substances to or from a cavity at an anatomic site. The catheter may stay in place such that samples may be taken from it. The catheter may stay at an anatomic site for days, weeks, or even longer. The organized tissue may be delivered from the catheter or remain within the catheter.

As used herein, “stent” refers to a self supporting structure which is delivered to an anatomic site by a catheter, or other means, to maintain a space in a normally non-open, or pathologically tightened or closed anatomic site. The stent may be formed of a self expanding material or of a shape-memory material which allows the stent to expand, creating such a space.

As used herein, “sleeve” refers to a biocompatible structure, having at least a first point for attachment and a second point for attachment. The sleeve is, in certain preferred embodiments, a porous, preformed structure. The sleeve can also be in the form of a mesh, net, or shape-memory material. The sleeve can be constructed from a material selected from the group including, but not limited to, polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched, woven, extruded or molded polytetrafluoroethylene, stretched, woven, extruded or molded polypropylene, stretched, woven, extruded or molded polyethylene, porous polyvinylidene fluoride, Angel Hair, silicon-oxygen-silicon matrices, polylsine and derivatives, copolymers and mixtures thereof The sleeve can also be constructed of natural materials including, but not limited to, collagen, extracellular matrix, intestinal mucosa, and metals including, but not limited to, stainless steel, tantalum, titanium and its alloys, and nitinol.

From the foregoing disclosure, it will be readily apparent to those skilled in the art, that is, those who are knowledgeable or experienced in this area of technology, that the present invention provides a significant technological advance. Substantial advantage is achieved by providing organized tissue to an anatomic site via a catheter. In particular, many of the cells of the organized tissue are not actively proliferating and are less likely to migrate away from an anatomic implant site. The implanted organized tissue can produce a bioactive compound that is needed in the organism, for example, producing therapeutic protein output levels for long time periods, offer controlled dosing, be placed in ectopic as well as homotopic sites, and be retrieved to terminate therapy. These and additional features and advantages of the invention disclosed here will be further understood from the following detailed disclosure of certain preferred embodiments.

### BRIEF DESCRIPTION OF THE DRAWINGS

Certain preferred embodiments are described in detail below with reference to the appended drawings wherein:

FIG. 1 is a schematic section view, shown partially broken away, of a catheter of the present invention, shown positioned within a blood vessel;

FIG. 2 is a schematic section view of a stent delivered by the catheter of FIG. 1 and positioned within the blood vessel of FIG. 1;

FIG. 3 is a schematic section view of an alternative embodiment of the catheter of FIG. 1;

FIG. 4 is a schematic section view of organized tissue positioned within the catheter of FIG. 1;

FIG. 5 is a schematic elevation view of an alternative embodiment of the organized tissue of FIGS. 2-4, shown anchored within a substrate;

FIG. 6 is a schematic section view of organized tissue housed in a sleeve positioned within the catheter of FIG. 1;

FIG. 7 is a graph showing a gluctose-lactase analysis of C2Cl2-hGH myoblasts encapsulated in PTFE tubes, and

FIG. 8 is a graph showing hGH secretion of C2Cl2-hGH myoblasts encapsulated in PTFE tubes.

The figures referred to above are not drawn necessarily to scale and should be understood to present a representation of the invention, illustrative of the principles involved. Some features depicted in the drawings have been enlarged or distorted relative to others to facilitate explanation and understanding. The same reference numbers are used in the drawings for similar or identical components and features shown in various alternative embodiments. Apparatus for the delivery of organized tissue as disclosed herein, will have configurations and components determined, in part, by the intended application and environment in which they are used.

### DETAILED DESCRIPTION OF CERTAIN PREFERRED EMBODIMENTS

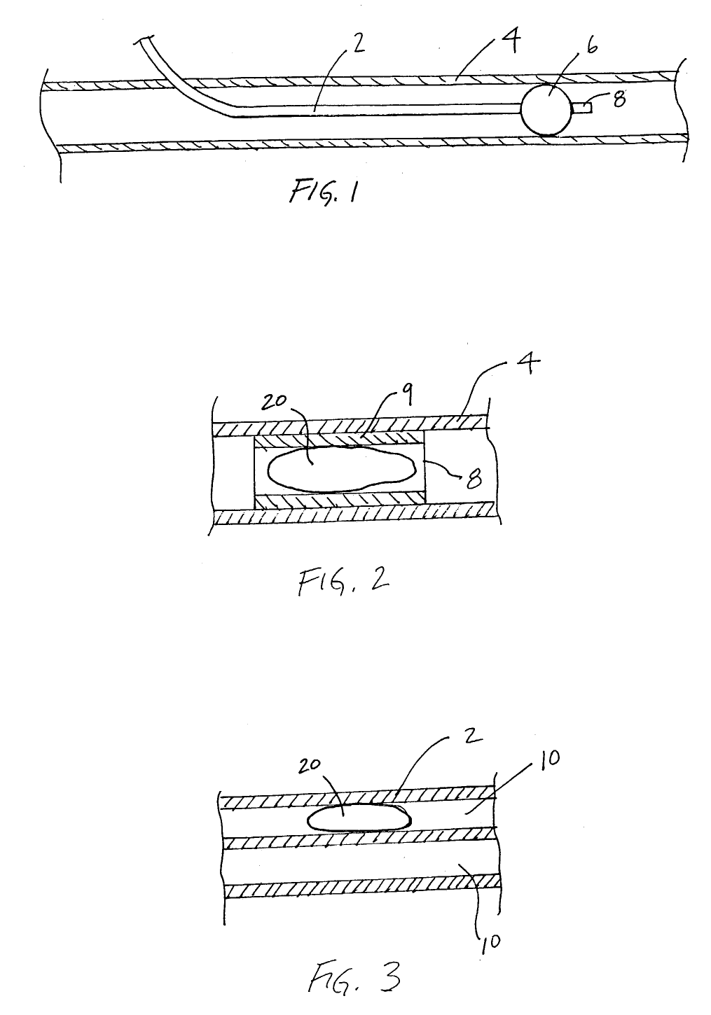

In accordance with certain preferred embodiments of the present invention, organized tissue can be delivered to biological sites for delivery of a bioactive compound such as a protein. The delivery of organized tissue may provide acute and/or chronic access to virtually all body cavities and fluid spaces as well as adjacent tissues or organs. In known fashion, as seen in FIG. 1, a catheter 2 may be introduced into tissue or vascular spaces, such as blood vessel 4, via transcutaneous needle puncture or an open cutdown procedure. Catheter 2 is shown being deployed within blood vessel 4, but it is to be appreciated that catheter 2 may be deployed in any tissue, organ, body cavity, joint space, or other suitable locations in the body in order to be effective against a disease. Catheter 2 may be advanced directly into open cavities or blood vessels. Balloon 6, seen proximate distal end 8 of catheter 2, may also be used to open and/or maintain spaces such as blood vessel 4. To access more difficult to reach areas, or even closed or stenotic areas, guide wires may first be placed in a desired site, over which sheaths and tissue dilators are placed in known fashion in order to open an area which will accommodate catheter 2. In certain instances, as seen in FIG. 2, a stent 9 may be deployed from catheter 2 or by other means into the desired site in order to maintain an open space. Organized tissue 20 can then be positioned within stent 9. Stent 9 may be formed from a variety of materials including, but not limited to, stainless steel, titanium, tantalum, and nitinol. In certain preferred embodiments, catheter 2 may be steered into surrounding tissue in order to deposit organized tissue therein.

In certain preferred embodiments, as seen in FIG. 3, catheter 2 may be provided with a plurality of lumens 10, with organized tissue 20 being positioned within one lumen 10. Multiple lumens 10 provide access for the delivery of additional components or the administration of fluids, adhesive glues or other materials.

Catheters as described above may be used in accordance with the present invention to provide acute access, e.g., to a tissue of interest, which may or may not be the same as the tissue of origin of the organized tissue, or to blood vessels and the abdominal cavity, as well as chronic indwelling, e.g., intensive care unit monitoring and chronic treatment using minimally invasive techniques. Access to various anatomic sites is available through the use of a catheter, including tissue (organ), subcutaneous, intraperitoneal, intraventricular (within the brain, within bone (in marrow spaces)) and intravascular locations. Catheters come in a range of sizes and shapes, any of which may be used for organized tissue therapy applications. In certain preferred embodiments, catheters have an internal diameter of approximately 100 μm to 2 cm and a length of approximately 5 mm to 500 cm. Catheters may be formed from a variety of materials, including polyurethane, polyethylene, polypropylene, fluorinated ethylene propylene, polytetrafluorethylene, or other suitable materials which will be readily apparent to those skilled in the art given the benefit of this disclosure.

Catheters can be used to deliver organized tissue to a variety of sites including, but not limited to: blood vessels; heart and pericardial space; tumors; gastrointestinal tract, joint spaces; ureters and kidney, brain parenchyma, ventricles, and intrathecal space; bone marrow space; eye; peritoneal cavity; thoracic cavity; liver; and ischemic muscle sites in the heart or limbs.

Catheters may also be used in conjunction with feeder and guide wires, trocars, introducers, dilators, balloons, stents and other mechanical medical devices in known fashion for delivery, positioning, deployment and anchorage of organized tissue. Endoscopic attachments may provide access to abdominal, thoracic and joint spaces. Light sources and fiberoptics may also be used in known fashion for direct visualization when delivering organized tissue. Radiographic guidance and localization can also be effected with catheters through the use of radiopaque markers. Additionally, catheters can be used to introduce liquid glues, e.g., fibrin, or sutures for anchorage in hard to reach locations.

Catheters are well known in the art and are described in U.S. Pat. No. 5,328,470 to Nabel et al. U.S. Pat. No. 5,409,470 to McIntyre et al.; U.S. Pat. No. 5,591,194 to Berthiaume; U.S. Pat. No. 5,713,861 to Vanarthos; U.S. Pat. No. 5,725,568 to Hastings; U.S. Pat. No. 5,728,067 to Enger; U.S. Pat. No. 5,759,191 to Barbere; U.S. Pat. No. 5,779,669 to Haussaguerre et al.; U.S. Pat. No. 5,797,870 to March et al.; and U.S. Pat. No. 5,823,995 to Fitzmaurice et al., the contents of which are incorporated herein by reference.

Stents are well known in the art and are described in U.S. Pat. No. 5,882,335 to Leone et al.; U.S. Pat. No. 5,713,949 to Jayaraman; U.S. Pat. No. 5,397,355 to Marin et al.; U.S. Pat. No. 5,035,706 to Giantureo et al.; U.S. Pat. No. 5,474,563 to Myler et al; and AmHeartJ 136:578-599, 1998 by Oesterle et al.

Organized Tissue:

An organized tissue useful in the invention is described in PCT/US97/00303, the contents of which are hereby incorporated by reference. Briefly, the organized tissue has an in vivo-like gross and cellular morphology of a tissue of interest and produces a protein of a type or produced in an amount not produced normally by the tissue of interest comprising: a plurality of cells, wherein at least a subset of cells comprise a foreign DNA sequence operably linked to a promoter and encoding a protein, wherein the cells form an organized tissue approximating the in vivo gross morphology of the tissue of interest and wherein the organized tissue is further comprised of post-mitotic cells; and wherein the protein is produced at detectable levels in the tissue.

As used herein, by a “bioactive compound” is meant a compound which influences the biological structure, function, or activity of a cell or tissue of a living organism; for example, a protein.

By “organized tissue” is meant a tissue formed in vitro from a collection of cells having a cellular organization and gross morphology similar to that of the tissue of origin for at least a subset of the cells in the collection. An organized tissue may include a mixture of different cells, for example, muscle (including but not limited to striated muscle, which includes both skeletal and cardiac muscle tissue), fibroblast, and nerve cells, but must exhibit the in vivo cellular organization and gross morphology that is characteristic of a given tissue including at least one of those cells, for example, the organization and morphology of muscle tissue may include parallel arrays of striated muscle tissue.

By “in vivo-like gross and cellular morphology” is meant a three-dimensional shape and cellular organization substantially similar to that of the tissue in vivo.

By “extracellular matrix components” is meant compounds, whether natural or synthetic compounds, which function as substrates for cell attachment and growth. Examples of extracellular matrix components include, without limitation, collagen, laminin, fibronectin, vitronectin, elastin, glycosaminoglycans, proteoglycans, and combinations of some or all of these components (e.g., Matrigel™, Collaborative Research, Catalog No. 40234).

An organized tissue useful according to the invention also may be attached to the surface of a substrate via tissue attachment surfaces. By “tissue attachment surfaces” is meant surfaces having a texture, charge or coating to which cells may adhere in vitro. Examples of attachment surfaces include, without limitation, stainless steel wire, VELCRO™, suturing material, titanium, ceramics, native tendon, covalently modified plastics (e.g., RGD complex), and silicon rubber tubing having a textured surface.

By “foreign DNA sequence” is meant a DNA sequence which differs from that of the wild type genomic DNA of the organism and may be extra-chromosomal, integrated into the chromosome, or the result of a mutation in the genomic DNA sequence.

By “substantially post-mitotic cells” is meant an organized tissue in which at least 50% of the cells containing a foreign DNA sequence are non-proliferative. Organized tissues including substantially post-mitotic cells also may be those in which at least 80%, 90% or even up to 99-100% of the cells containing a foreign DNA sequence are non-proliferative. Cells of an organized tissue retaining proliferative capacity may include cells of any of the types included in the tissue. For example, in striated muscle organized tissues such as skeletal muscle organized tissues, the proliferative cells may include muscle stem cells (i.e., satellite cells) and fibroblasts.

I. Production of an Organized Tissue and Transfer to Catheter

An organized tissue having in vivo-like gross and cellular morphology may be produced in vitro from the individual cells of a tissue of interest. As a first step in this process, disaggregated or partially disaggregated cells are mixed with a solution of extracellular matrix components to create a suspension. This suspension is then placed in a vessel having a three dimensional geometry which approximates the in vivo gross morphology of the tissue and includes tissue attachment surfaces coupled to the vessel. The cells and extracellular matrix components are then allowed to coalesce or gel within the vessel, and the vessel is placed within a culture chamber and surrounded with media under conditions in-which the cells are allowed to form an organized tissue connected to the attachment surfaces.

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung. More specific examples include skeletal and cardiac (i.e., striated), and smooth muscle, stratified or lamellar bone, and hyaline cartilage. Where the tissue includes a plurality of cell types, the different types of cells may be obtained from the same or different organisms, the same or different donors, and the same or different tissues. Moreover, the cells may be primary cells or immortalized cells. Furthermore, all or some of the cells of the tissue may contain a foreign DNA sequence which mediates the production of a bioactive compound.

Alternative methods of production of organized tissue include the use of dynamic mechanical cell stimulation as described in U.S. Pat. No. 4,940,853. Other suitable methods of production of organized tissue will become readily apparent to those skilled in the art, given the benefit of this disclosure.

The composition of the solution of extracellular matrix components will vary according to the tissue produced. Representative extracellular matrix components include, but are not limited to, collagen, laminin, fibronectin, vitronectin, elastin, glycosaminoglycans, proteoglycans, and combinations of some or all of these components (e.g., Matrigel™, Collaborative Research, Catalog No. 40234). In tissues containing cell types which are responsive to mechanical forces, the solution of extracellular matrix components preferably gels or coalesces such that the cells are exposed to forces associated with the internal tension in the gel.

Culture conditions will also vary according to the tissue produced. Methods for culturing cells are well known in the art and are described, for example, in Skeletal Cell Culture: A Practical Approach, (R. I. Fveshney, ed. IRL Press, 1986). In general, the vessel containing a coalesced suspension of cells and extracellular matrix components is placed in a standard culture chamber (e.g., wells, dishes, or the like), and the chamber is then filled with culture medium until the vessel is submerged. The composition of the culture medium is varied, for example, according to the tissue produced, the necessity of controlling the proliferation or differentiation of some or all of the cells in the tissue, the length of the culture period and the requirement for particular constituents to mediate the production of a particular bioactive compound. The culture vessel may be constructed from a variety of materials in a variety of shapes as described below.

In the embodiment of the invention wherein the tissue having an in vivo-like gross and cellular morphology is grown in vitro, the vessel in which the tissue is grown also includes tissue attachment surfaces which are an integral part of or coupled to the vessel. Such a vessel may be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. The tissue attachment surfaces (e.g., stainless steel mesh, VELCRO™, or the like) are coupled to the vessel and positioned such that as the tissue forms in vitro the cells may adhere to and align between the attachment surfaces. The tissue attachment surfaces may be constructed from a variety of materials which are compatible with the culturing of cells and tissues (e.g., capable of being sterilized, or having an appropriate surface charge, texture, or coating for cell adherence).

The tissue attachment surfaces may be coupled in a variety of ways to an interior or exterior surface of the vessel. Alternatively, the tissue attachment surfaces may be coupled to the culture chamber such that they are positioned adjacent the vessel and accessible by the cells during tissue formation. In addition to serving as points of adherence, in certain tissue types (e.g., muscle), the attachment surfaces allow for the development of tension by the tissue between opposing attachment surfaces. Moreover, where it is desirable to maintain this tension in vivo, the tissue adhered to the tissue attachment surfaces may be transferred to a catheter according to the invention and the catheter utilized to implant the tissue into an organism.

A. Production of a Skeletal Muscle Organized Tissue and Transfer to Catheter

Using the method as generally described above, a skeletal muscle organized tissue having an in vivo-like gross and cellular morphology was produced in vitro as described in Shansky et al., In Vitro Cell Developmental Biology, vol. 33, pages 659-661, 1997. During skeletal muscle development embryonic myoblasts proliferate, differentiate, and then fuse to form multi-nucleated myofibers. Although the myofibers are non-proliferative, a population of muscle stem cells (i.e., satellite cells), derived from the embryonic myoblast precursor cells, retain their proliferative capacity and serve as a source of myoblasts for muscle regeneration in the adult organism. Therefore, either embryonic myoblasts or adult skeletal muscle stem cells may serve as one of the types of precursor cells for in vitro production of a skeletal muscle organized tissue.

To produce skeletal muscle cells capable of secreting a bioactive compound, primary rat or avian cells or immortalized murine cells secreting recombinant human growth hormone, were suspended in a solution of collagen and Matrigel™ which was maintained at 4° C. to prevent gelling. The cell suspension was then placed in a semi-cylindrical vessel with tissue attachment surfaces coupled to an interior surface at each end of the vessel. The vessel was positioned in the bottom of a standard cell culture chamber. Following two to four hours of incubation at 37° C., the gelled cell suspension was covered with fresh culture medium (renewed at 24 to 72 hour intervals) and the chamber containing the suspended cells was maintained in a humidified 5% CO₂ incubator at 37° C. throughout the experiment.

Between the second and sixth day of culture, the cells were found to be organized to the extent that they spontaneously detached from the vessel. At this stage, the cells were suspended in culture medium while coupled under tension between tissue attachment surfaces positioned at either end of the culture vessel. During the subsequent ten to fourteen days, the cells formed an organized tissue containing skeletal myofibers aligned parallel to each other in three dimensions. The alignment of the myofibers and the gross and cellular morphology of the organized tissue were similar to that of in vivo skeletal muscle.

To carry out the above method, an apparatus for organized tissue formation was constructed from silastic tubing and either VELCRO™ or metal screens as follows. A section of silastic tubing (approximately 5 mm I.D., 8 mm O.D., and 30 mm length) was split in half with a razor blade and sealed at each end with silicone rubber caulking. Strips of VELCRO™ (loop or hook side, 3 mm wide by 4 mm long) or L-shaped strips of stainless steel screen (3 mm wide by 4 mm long by 4 mm high) were then attached with silicone rubber caulking to the interior surface of the split tubing near the sealed ends. The apparatus was thoroughly rinsed with distilled/deionized water and subjected to gas sterilization.

Skeletal muscle organized tissues were produced in vitro from a C2Cl2 mouse skeletal muscle myoblast cell line stably co-transfected with recombinant human growth hormone-expressing and β-galactosidase-expressing β-gal) constructs. Dhawan et al., 1991, Science 254:1509-1512. Cells were plated in the vessel at a density of 1-4×10⁶ cells per vessel in 400 μl of a solution containing extracellular matrix components. The suspension of cells and extracellular matrix components was achieved by the following method. The solution includes 1 part Matrigel™ (Collaborative Research, Catalog No. 40234) and 6 parts of a 1.6 mg/ml solution of rat tail Type I collagen (Collaborative Research, Catalog No. 40236). The Matrigel™ was defrosted slowly on ice and kept chilled until use. The collagen solution was prepared just prior to cell plating by adding to lyophilized collagen, growth medium (see constituents below), and 0.1N NaOH in volumes equivalent to 90% and 10%, respectively, of the volume required to obtain a final concentration of 1.6 mg/ml and a pH of 7.0-7.3. The collagen, sodium hydroxide and growth medium were maintained on ice prior to and after mixing by inversion.

Freshly centrifuged cells were suspended in the collagen solution by trituration with a chilled sterile pipet. Matrigel™ was subsequently added with a chilled pipet and the suspension was once again mixed by trituration. The suspension of cells and extracellular matrix components was maintained on ice until it was plated in the vessel using chilled pipet tips. The solution was pipetted and spread along the length of the vessel, taking care to integrate the solution into the tissue attachment surfaces. The culture chamber containing the vessel was then placed in a standard cell culture incubator, taking care not to shake or disturb the suspension. The suspension was allowed to gel, and after 2 hours the culture chamber was filled with growth medium such that the vessel was submerged.

For a period of three days the cells were maintained on growth medium containing DMEM-high glucose (GIBCO-BRL), 5% fetal calf serum (Hyclone Laboratories), and 1% penicillin/streptomycin solution (final concentration 100 units/ml and 0.1 μg/ml, respectively). On the fourth day of culture, the cells were switched to fusion medium containing DMEM-high glucose, 2% horse serum (Hyclone Laboratories), and 100 units/ml penicillin for a period of 4 days. On the eighth day of culture, the cells were switched to maintenance medium containing DMEM-high glucose, 10% horse serum, 5% fetal calf serum, and 100 units/ml penicillin for the remainder of the experiment. Before the organized tissues were ready for implantation, some were cultured in maintenance media containing 1 μg/ml of cytosine arabinoside for the final four to eight days. Treatment with cytosine arabinoside eliminated proliferating cells and produced organized tissues including substantially post-mitotic cells.

The cell-extracellular matrix gel (cell-gel) formed in vitro from these stably transfected C2Cl2 cells reveals cell growth in parallel arrays of highly organized and longitudinally oriented myofibers in mammalian skeletal muscle organized tissues following three weeks of culturing. Using mechanical means, the skeletal muscle organized tissue is then transferred so as to lie within the distal end of a catheter, and the catheter containing the organized tissue is then ready for use in transferring the organized tissue into an organism.

II. Production of an Organized Tissue in a Catheter and/or Sleeve

An organized tissue may be grown in a catheter as follows. Organized tissue cells in a biocompatible physiological buffered solution are injected in a catheter having a desired porosity. The catheter is then placed in a petri dish containing a suitable media solution and maintained under controlled conditions for a number of days. The solution in which the petri dish is maintained may be periodically changed.

Thus, a kit according to the invention will include a device of the invention, comprising a catheter containing one or more organized tissues, a biocompatible physiological tissue culture media and buffer in which the organized tissue is maintained within the catheter for from hours to days to weeks without significant loss of viability and bioactivity, and packaging materials therefor. The biocompatible physiological buffer includes, minimally, tissue culture media and phosphate buffered saline, and also may include additional components such as growth factors and serum.

Organized tissue, in certain preferred embodiments, can be provided in a size of approximately 0.5 mm long by 100 μm wide to approximately 100 cm long by 100 cm wide. The organized tissue may be placed at ectopic as well as homotopic sites since it is preformed tissue. One or more (2-5, 10, 20 or even more) organized tissues may be present in a catheter or introduced by several catheters, depending upon the amount of tissue that is desired at a given anatomic site or for a given biological effect, for instance a dosing effect where the organized tissue producing a bioactive compound that is required at a given dosage in the organism.

Use of Device to Deliver Organized Tissue and Bioactive Compound

A bioactive compound may be delivered to an organism using a device according to the invention, i.e., a catheter containing an organized tissue that produces the bioactive compound, and after catheterization, implanting the organized tissue into the organism. The organized tissue may be pre-loaded or formed in a sleeve which is placed in the catheter for delivery.

A variety of bioactive compounds may be delivered by this method, and they may function through intracellular (i.e., within the cells of the organized tissue), endocrine, autocrine, or paracrine mechanisms. Moreover, the organized tissue may deliver multiple bioactive compounds either simultaneously or sequentially (e.g., one bioactive compound mediates the delivery of another). Liberation of the bioactive compound from the cells of the organized tissue may occur by either passive or active processes (e.g., diffusion or secretion).

For example, the bioactive compound may be a hormone, growth factor, cytokine or the like which is produced and liberated by the cells of the organized tissue to act locally or systemically on host tissues. Alternatively, the bioactive compound may function within the cells or on the surface of the cells of the organized tissue to enhance the uptake or metabolism of compounds from the host tissue or circulation (e.g., lactic acid, low density lipoprotein). Where the organized tissue serves as a functional and structural adjunct to the host tissue, delivery of growth factors by autocrine or paracrine mechanisms may enhance the integration of the organized tissue into host tissues. Similarly, where multiple bioactive compounds are produced by the organized tissue, autocrine delivery of one of the bioactive compounds may be used to regulate the production of one or more of the other bioactive compounds.

The organized tissue may be implanted at a desired anatomical location within the organism. For example, the organized tissue may be implanted in the same or a different tissue from the tissue of origin of at least one of the individual cells. The location of implantation depends, in part, upon the identity of the particular bioactive compound to be delivered. For example, an organized tissue acting as an endocrine organ may be implanted in or adjacent a highly vascularized host tissue. Alternatively, an organized tissue acting as a paracrine organ is preferably implanted in or adjacent to the host tissue to which the bioactive compound is to be delivered.

The organized tissue may be implanted by attachment to a host tissue or as a free floating tissue. In addition, attached organized tissues may be implanted with or without the tissue attachment surfaces used for in vitro tissue formation. Tissues responsive to mechanical forces are preferably implanted by attaching directly to the host tissue or by implanting the organized tissue coupled to the attachment surfaces so that the organized tissue is exposed to mechanical forces in vivo. For example, skeletal muscle organized tissues are preferably implanted by attachment to the host tissue under tension along a longitudinal axis of the organized tissue. Moreover, the organized tissues may be permanently or temporarily implanted. Permanent implantation may be preferred, for example, where the organized tissue produces a bioactive compound which corrects a systemic metabolic error (e.g., delivery of insulin to treat diabetes), whereas temporary implantation may be preferred where only transient delivery of a bioactive compound is desired (e.g., delivery of a growth factor to enhance wound healing). Furthermore, because organized tissues may be implanted, removed, and maintained in vitro, bioactive compounds may be delivered intermittently to the same or a different location in the organism. For example, a skeletal muscle organized tissue produced from the cells of a human patient (e.g., an autograft or allograft) may be implanted at a first anatomical location for a defined period and subsequently implanted at a second location at or after the time of removal.

At least some of the cells of the organized tissue contain a foreign DNA sequence. The foreign DNA sequence may be extra-chromosomal, integrated into the genomic DNA of the organized tissue cell, or may result from a mutation in the genomic DNA of the organized tissue cell. In addition, the cells of the organized tissue may contain multiple foreign DNA sequences. Moreover, the different cells of the organized tissue may contain different foreign DNA sequences. For example, in one embodiment, a skeletal muscle organized tissue may include myofibers containing a first foreign DNA sequence and fibroblasts containing a second foreign DNA sequence. Alternatively, the skeletal muscle organized tissue could include myoblasts from different cell lines, each cell line expressing a foreign DNA sequence encoding a different bioactive compound. These “mosaic” organized tissues allow the combined and/or synergistic effects of particular bioactive compounds to be exploited. For example, myoblasts expressing growth hormone may be combined with myoblasts expressing an insulin-like growth factor to produce organized tissues useful in stimulating muscle growth/regeneration. Similarly, myoblasts expressing a bone morphogenetic protein may be combined with myoblasts expressing a parathyroid hormone to produce organized tissues useful in stimulating bone and cartilage growth/regeneration. A bioactive compound according to the invention can include, but is not limited to erythropoietin (EPO), insulin-like growth factor-1 (1GF-1), VEGF, β-galactosidase, cytokine, growth hormone, and bone morphogenetic protein.

The foreign DNA sequence may encode a protein which is the bioactive compound. The protein is produced by the cells and liberated from the organized tissue. Alternatively, the DNA sequence may encode an enzyme which mediates the production of a bioactive compound or a cell surface protein which enhances the uptake and metabolism of compounds from the host tissue or circulation (e.g., lactic acid or low density lipoproteins). The DNA sequence may also encode a DNA binding protein which regulates the transcription of the sequence encoding a bioactive compound or an antisense RNA which mediates translation of the mRNA for the bioactive compound. The DNA sequence may also bind trans-acting factors such that the transcription of the sequence (i.e., foreign or native) encoding the bioactive compound is enhanced (e.g., by disinhibition). Furthermore, the foreign DNA sequence may be a cis-acting control element such as a promoter or an enhancer coupled to a native or foreign coding sequence for the bioactive compound or for an enzyme which mediates the production of the bioactive compound. Thus, the foreign DNA sequence may be expressible in the cell type into which it is introduced and may encode a protein which is synthesized and which may be secreted by such cells. Alternatively, the foreign DNA sequence may be an element that regulates an expressible sequence in the cell.

In order to correlate the delivery dose of an organized tissue implanted in vivo for treatment according to the invention, organized tissue protein secretion levels can be varied by engineering a protein-producing organized tissue with different numbers of protein-secreting cells. In addition, varying numbers of organized tissues can be implanted and levels of bioactive compound determined. For example, where one to four organized tissues producing recombinant human growth hormone (rhGH) has been implanted per animal, and corresponding increase in the level of bioactive compound was found. A correlation was found of in vivo rhGH serum levels from rhGH levels secreted in vitro. A linear relationship was found to exist for the amount of rhGH secreted by rhGH-producing organized tissues preimplantation and postimplantation.

External control of the organized tissue contained within the catheter, sleeve and or stent is possible. Small molecules can pass through pores formed in the catheter, sleeve and or stent. A porosity size will be selected based on the molecule size of the substance to be passed through the pores. For example, antibiotics such as tetracycline, insect steroids, doxycycline, rapomycin, or other molecules may be used which will diffuse across the catheter, sleeve and or stent to regulate production of a protein from the organized tissue.

In one embodiment of the invention, the pore size of the material which forms the catheter, sleeve and/or stent permits passage of doxycycline (DOX) 1 μg/ml in the culture medium immediately surrounding the catheter, sleeve and/or stent (the organoid is engineered to contain the EPO gene under control of the DOX-activated promoter). After approximately 4 days, DOX-stimulated organoids will secrete approximately 4±0.2 μEPO/day in vitro. After introduction into the body, the organoid will maintain the same level of secretion in vivo. Any small molecule gene regulatory system may be used, for example, the gene of interest (e.g., the EPO gene above) may be placed under control of a small-molecule-sensitive promoter.

The invention is applicable to therapies in which one or more bioactive compounds are delivered to an organism, for example, a mammal in therapeutically effective levels. A therapeutic gene is one which is expressible in a mammalian, preferably a human, cell and encodes RNA or a polypeptide that is of therapeutic benefit to a mammal, preferably a human. A vector may also include marker genes, such as drug resistance genes, the β-galactosidase gene, the dihydrofolate reductase gene, and the chloramphenicol acetyl transferase gene. A therapeutic effect is evident, for example, where the therapeutic gene encodes a product of physiological importance, such as replacement of a defective gene or an additional potentially beneficial gene function, is expected to confer long term genetic modification of the cells and be effective in the treatment of disease.

The dosages of a bioactive compound administered according to the invention will vary from patient to patient; a “therapeutically effective dose” will be determined by the level of enhancement of function of the transferred genetic material balanced against any risk or deleterious side effects. Monitoring levels of gene introduction, gene expression and/or the presence or levels of the encoded product will assist in selecting and adjusting the dosages administered. Generally, a composition including a bioactive compound-producing organized tissue according to the invention will be administered in a single dose (per time period in which the organized tissue implant is judged to be effective in producing the bioactive compound), such that the bioactive compound is produced in the mammal in the range of 1 pg-100 mg/kg body weight, preferably in the range of 100 ng-10 μg/kg body weight, depending upon the nature of the bioactive compound, its half-life, and its biological effect. By “therapeutically effective amount” also is meant capable of attenuating the clinical symptoms of a disease or a clinical deficiency associated with a disease in an organism by at least 5-10%, preferably 20-30% and more preferably 35-100%, as compared to an untreated organism.

The devices and methods of disease treatment according to the invention, is suitable for treating diseases including but not limited to blood disorders, bone and joint disorders, cancer, cardiovascular disorders, endocrine disorders, immune disorders, infectious diseases, wasting disorders, neurological disorders and skin disorders. Treatment of tissue wasting cachexia may be achieved using a bioactive compound which is a growth hormone, insulin and/or insulin-like growth factor, treatment of a neurological disorder may be achieved where the bioactive compound is a nerve growth factor (e.g. NGF, CNTF, or bFGF); treatment of a skin disorder such as a ulcer may be achieved where the bioactive compound is EGF, or wound healing where the bioactive compound is TGF-β or PDGF; treatment of cardiovascular disorders may be achieved where the bioactive compound is vascular endothelial factor or basic fibroblast growth factor or insulin-like growth factor 1. Treatment of cancer may be achieved using a cytokine (IL-2, IL-12) or anti-angiogenic agent (endostatin).

### Use and Advantages

Organized tissue delivered via a catheter to biological sites provides numerous advantages. Organized tissue constructs are formed from stable, post-mitotic cells which are less likely than proliferating cells to migrate away from their implantation site. The organized tissue can be stably altered to produce therapeutic proteins over long periods of time, offer the ability to monitor protein output levels prior to implantation and offer controlled dosing based on the number of cells or the number of organized tissues implanted. Organized tissue, being preformed or pre-organized, can be placed in ectopic as well as homotopic sites and tissues and can be retrieved to terminate therapy. The protein output can be determined prior to implantation.

Organized tissue, in accordance with preferred embodiments of the present invention, can provide diagnostic, or sensing, capabilities, e.g., detecting tissue byproduct, sensing pH, monitoring the motion or mechanical activity of other tissue. Organized tissue, in accordance with preferred embodiments of the present invention may also be used to treat fetuses diagnosed with certain abnormalities. Other treatments for which organized tissue delivered in accordance with the present invention will be applicable will become readily apparent to those skilled in the art, given the benefit of this disclosure.

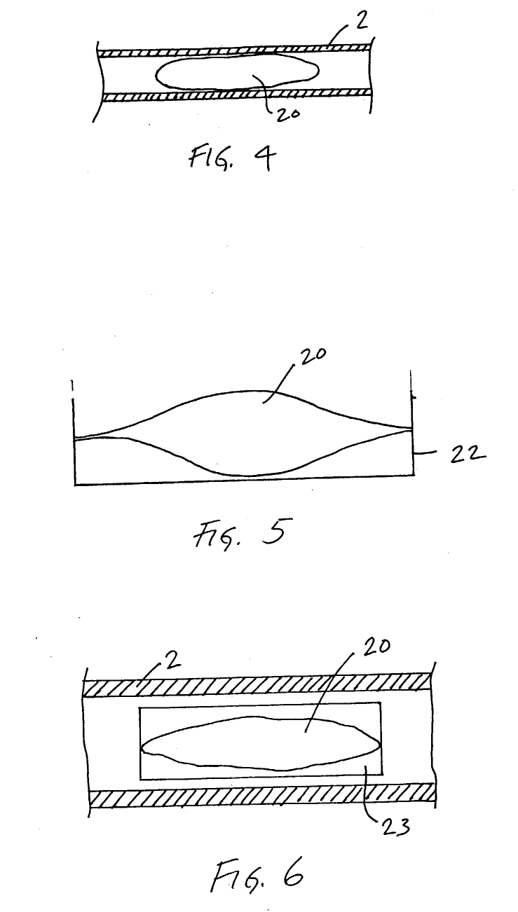

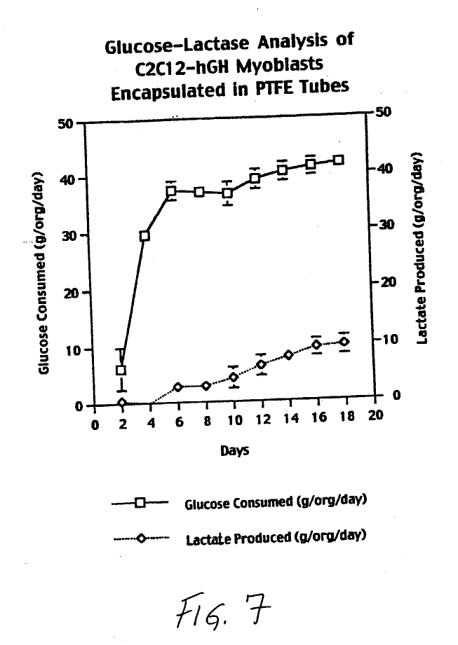

In use, as seen in FIG. 4, the preformed organized tissue 20 is placed in catheter 2 and delivered to the desired biological site, such as blood vessel 4 shown in FIG. 1. Depending on the desired treatment, organized tissue may, in certain preferred embodiments, be grown within catheter 2 or on other devices, e.g., sleeves 23 (seen in FIG. 6 below), wire stents or balloons carried within catheter 2. In other preferred embodiments, the portion of catheter 2 containing organized tissue 20 may be clipped off and left at the desired anatomic site for treatment. Organized tissue 20 may be retrieved to end therapy through the use of forceps, baskets, or other mechanical devices well known to those skilled in the art.

In another preferred embodiment, a kit is used for the treatment of disease. The kit comprises catheter 2, organized tissue 20, and buffer 30 for maintaining organized tissue 20. Buffer 30 may be comprised of tissue culture media, phosphate buffered saline or other suitable components for maintaining the viability of organized tissue 20 Organized tissue 20 is delivered to a desired biological site via catheter 2 for treatment of a disease. The kit may also contain sleeve 23, stent 8 and/or balloon 6 which are delivered via catheter 2 to the desired biological site. As noted above, catheter 2 may contain a plurality of lumens 10 or sleeves 23.

In another preferred embodiment, shown in FIG. 5, organized tissue 20 is anchored at opposite ends thereof to substrate 22. Substrate 22 may be formed of plastic, metal, mesh, or other suitable material. Substrate 22 containing organized tissue 20 is then placed in a catheter and inserted via a catheter in the manner described above. Anchoring organized tissue 20 to substrate 22 advantageously introduces tension into the organized tissue which increases the amount of bioactive compound produced by the tissue.

The delivery of organized tissue offers significant advantages. Organized tissue constructs are formed from stable, postmitotic cells, and many of the cells of the organized tissue are therefore are less likely to migrate away from the biological implant site. Therapeutic output can be determined prior to implantation. The organized tissue constructs can produce a bioactive compound that is needed in the organism and can, for example, produce therapeutic protein output levels for long time periods, offer controlled dosing based on the number of cells or the number of organized tissues implanted, be placed in ectopic as well as homotopic sites and tissues, be retrieved to terminate therapy when desired, and are defined structures which can be loaded into a variety of catheters and stents to be delivered and deployed.

### EXAMPLE 1

### Formation of Organized Tissue in a Catheter

An experiment was conducted to assess the long-term viability, GH output, and ultrastructure of C2Cl2 cells transfected with the hGH gene in a microporous, ePTFE tube.

Tube Construction: Tubes of ePTFE with a pore size of approximately 20 microns were cut into 3 cm segments. On one end of the tube, a stainless steel screw was inserted and loosely tied with 6-0 silk suture. On the other end, a gas-line screw, which has an open channel running from end to end, was inserted and loosely tied with 6-0 silk suture. Upon insertion of both screws, the total open volume within the tube was roughly 0.295 cm³.

Cell Suspension: C2Cl2 cells transfected with hGH were grown in Growth Media (20% serum) until confluence in a T-175 flask. The cells were trypsinized and counted. 5.1 million cells were resuspended in a 1.5 mL solution. This solution consisted of 1/7 Matrigel and 6/7 collagel (Type I RTT collagen, NaOH, and C2GM).

Cell Injection: The cell suspension described above was injected into a total of 4 tubes via a 3.0 cc syringe and 20 GI needle. Each tube was filled with cell suspension containing 1×10⁶ cells, and then the open channel was sealed using Light Cured Resin (manufactured by Ablestik of California). The tube was then placed into a 60 mm petri dish which was filled with 15 mL of C2GM. The dishes were kept in an incubator at 37° C. and 10%CO₂.

Maintenance and Sample Collection: The media was changed every two days. During the change, 2×1 mL aliquots were saved for GH and glucose-lactase analysis. The tubes were kept in GM for the first four days, then FM was used for the next four days, and MM was used for the rest of the study (the tubes were kept for a total of 18 days).

Glucose-Lactase Analysis: Samples were tested using a YSI Glucose-Lactase Analyzer Model 2000 (manufactured by YSI Incorporated) which provided glucose and lactase concentrations in g/L.

hGH Analysis: The hGH concentration was assessed using a human growth hormone RIA assay for transient gene expression (from Nichols Institute Diagnostics, California), which gave results in ng/mL.

Results: Glucose-lactase analysis is summarized in the graph shown in FIG. 7. Glucose levels rose rapidly through the first four days, and then plateaued thereafter showing cell fusion. This is due to the use of Fusion Media (2% serum) from days 4 through 8 to slow proliferation and stimulate myofiber formation via myoblast fusion.

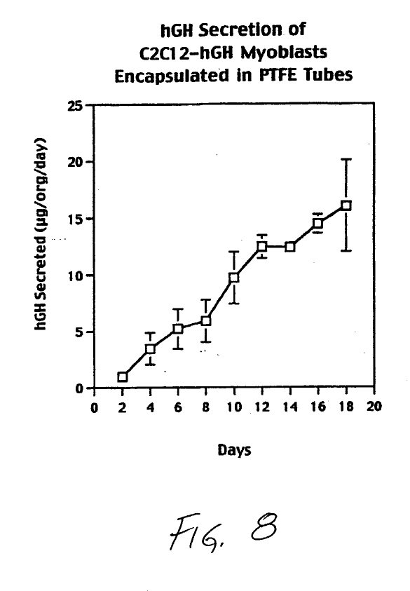

Growth hormone analysis through day 18, as seen in the graph shown in FIG. 8, indicated that hGH was being released from the organoids into the media. At day 8, the hGH output averaged approximately 5.9 μg hGH/10⁶ cells/day which is similar for 1×10⁶ cells grown in open troughs where output remains at that level. In contrast, cells grown in micro-porous tubes showed increasing GH output levels. For example, at day 16 the output rose to roughly 14.4 μg hGH/10⁶ cells/day which is a three fold higher level than for cells grown in open troughs.

In light of the foregoing disclosure of the invention and description of the preferred embodiments, those skilled in this area of technology will readily understand that various modifications and adaptations can be made without departing from the true scope and spirit of the invention. All such modifications and adaptations are intended to be covered by the following claims.

We claim:

1. A device for delivery of a tissue to an organism, said device comprising, in combination: a catheter; and an organized tissue capable of being contained within said catheter, wherein said organized tissue comprises a plurality of cells, said organized tissue having an in vivo-like gross and cellular morphology and producing a bioactive compound of a type or produced in an amount not normally produced by a tissue of interest.

2. A device for delivery of a bioactive compound to an organism, said device comprising, in combination: a catheter; and an organized tissue that produces said bioactive compound and that is capable of being contained within said catheter, wherein the organized tissue is delivered to an anatomic site of said organism, via the catheter, wherein said organized tissue comprises a plurality of cells, said organized tissue having an in vivo-like gross and cellular morphology and producing a bioactive compound of a type or produced in an amount not normally produced by a tissue of interest.

3. The device of claim 1 or 2, wherein said organized tissue comprises first and second attachment sites and is anchored via said first and second attachment sites to the surface of a substrate, wherein said organized tissue anchored to said substrate is contained within said catheter.

4. The device of claim 1 or 2, further comprising a stent for insertion within a cavity of the catheter.

5. The device of claim 4, wherein the organized tissue is deposited within the stent.

6. The device of claim 4, further comprising a balloon for insertion within a cavity via the catheter.

7. The device of claim 1 or 2, wherein the organized tissue is grown within the catheter.

8. The device of claim 1 or 2, wherein the organized tissue is deposited in a sleeve for insertion within a cavity via the catheter.

9. The device of claim 1 or 2, wherein the catheter has a plurality of lumens.

10. The device of claim 2, wherein the organized tissue is anchored in vivo at the anatomic site.

11. The device of claim 10, wherein the organized tissue is anchored with one of sutures, adhesive, and barbs.

12. The device of claim 1 or 2, wherein the organized tissue is anchored to a substrate.

13. The device of claim 1 or 2, wherein said organized tissue does not remain in the catheter following said delivery.

14. The device of claim 1 or 2, wherein said organized tissue further comprises substantially post-mitotic cells.

15. A method of delivering a tissue to an organism, comprising the steps of: providing a catheter; growing organized tissue within the catheter; and delivering the organized tissue capable of being contained within the catheter to an organism via the catheter, wherein said organized tissue comprises a plurality of cells, has an in vivo-like gross and cellular morphology and produces a bioactive compound of a type or produced in an amount not normally produced by a tissue of interest.

16. The method of claim 15, wherein said organized tissue further comprises substantially post-mitotic cells.

17. A method of delivering a bioactive compound to an organism comprising the steps of providing a catheter; growing organized tissue that produces said bioactive compound within the catheter; and delivering the organized tissue capable of being contained within the catheter to an anatomic site via the catheter, wherein said organized tissue comprises a plurality of cells, has an in vivo-like gross and cellular morphology and produces a bioactive compound of a type or produced in an amount not normally produced by a tissue of interest.

18. The method of claim 15 or 17, further comprising the step of retrieving the organized tissue from said organism.

19. The method of claim 15 or 17, wherein said organized tissue does not remain in the catheter following said delivery.

20. The method of claim 17, wherein said organized tissue further comprises substantially post-mitotic cells.
